(19) United States
(12) Patent Application Publication
(10) Pub. No.: US 2024/0009433 A1
(43) Pub. Date: Jan. 11, 2024

(54) METHODS FOR CONTINUOUS ORAL DRUG DELIVERY

(71) Applicant: SynAgile Corporation, San Jose, CA (US)

(72) Inventor: Adam HELLER, Austin, TX (US)

(21) Appl. No.: 18/025,244

(22) PCT Filed: Sep. 13, 2021
(86) PCT No.: PCT/US2021/050024

§ 371 Date: Mar. 8, 2023

Related U.S. Application Data
(60) Provisional application No. 63/077,086, filed on Sep. 11, 2020.

(51) Int. Cl. A61M 31/00; A61K 9/00; A61J 7/00

(52) U.S. Cl. CPC A61M 31/002; A61K 9/0053; A61J 7/0092; A61M 2210/0625; A61M 2205/3334

(57) ABSTRACT

The invention features methods for continuously administering a drug by oral infusion (e.g., via a drug delivery device anchored in the mouth for continuously administering a pharmaceutical composition), and pre-screening subjects and, if necessary, treating subjects with H. pylori to improve the PK performance of the continuous intraoral administration in the subject.

### FIELD OF THE INVENTION

The invention features methods for continuously administering a drug by oral infusion (e.g., via a drug delivery device anchored in the mouth for continuously administering a pharmaceutical composition), and pre-screening subjects and, if necessary, treating subjects with H. pylori to improve the PK performance of the continuous intraoral administration in the subject.

### BACKGROUND

This invention features methods for continuous or semi-continuous drug administration via the oral route. It is an aim of this invention to solve several problems related to drugs with short physiological half-lives (e.g., shorter than 8 hours, 6 hours, 4 hours, 2 hours, 1 hour, 30 min, 20 min or 10 min) and/or narrow therapeutic windows of drugs that are currently dosed multiple times per day: it is inconvenient to take a drug that must be dosed multiple times per day or at night, the drug's pharmacokinetics and efficacy may be sub-optimal, and side effects may increase in frequency and/or severity. Continuous or semi-continuous administration can be particularly beneficial for drugs with a short half-life (e.g., in the plasma), and/or short persistence of the drug's physiological effect, and/or a narrow therapeutic window, such as levodopa (LD), muscle relaxants (e.g., baclofen for managing spasticity), anti-epileptics (e.g., oxcarbazepine, topiramate, lamotrigine, gabapentin, carbamazepine, valproic acid, levetiracetam, pregabalin), parasympathornimetics (e.g., pyridostigmine) and sleep medications (e.g., zaleplon). Continuous or semi-continuous infusion in the mouth can provide for lesser fluctuation in the concentration of a drug in an organ or fluid, for example in the blood or plasma. Convenient, automatic administration of a drug can also increase patient compliance with their drug regimen, particularly for patients who must take medications at night and for patients with dementia.

Medical conditions managed by continuously orally administered drugs include Parkinson's disease, spasticity, muscular weakness, bacterial infections, cancer, pain, organ transplantation, disordered sleep, epilepsy and seizures, anxiety, mood disorders, post-traumatic stress disorder, arrhythmia, hypertension, heart failure, dementia, allergies, and diabetic nephropathy.

Most drugs intended for oral administration have been formulated as solids (e.g., pills, tablets), solutions, or suspensions that are administered once or several times per day. Such drugs are not formulated to meet the requirements of continuous or semi-continuous, constant-rate, intra-oral administration. For example, many suspensions and solutions have been formulated in relatively large daily volumes that don't fit in the mouth without interfering with its functions, particularly with speech, and/or in formulations that are physically or chemically unstable over the course of a day at body temperature; and pills and tablets have rarely been formulated in units and dosage amounts appropriate for dosing frequently throughout the day.

Large quantities of drug must be administered to treat some diseases. For example, 1,000 mg of levodopa is a typical daily dose administered to patients with advanced Parkinson's disease. In order to continuously administer such large quantities of drug into the mouth in a fluid volume that will fit comfortably in the mouth (typically less than 5 mL) for many hours, it is sometimes necessary to employ concentrated, often viscous, fluid formulations of the drug, such as drug-comprising pastes, that can be continuously extruded into the mouth. Use of viscous fluids can provide the small volumes, high concentrations, uniform drug dispersion, storage stability, and operational stability desired for the drugs and methods of the invention. Consequently, it is often necessary to employ miniaturized pumps tailored to provide the pressures required to pump the viscous fluids.

As a specific example, Parkinson's disease (PD) is characterized by the inability of the dopaminergic neurons in the substantia nigra to produce the neurotransmitter dopamine. PD impairs motor skills, cognitive processes, autonomic functions, and sleep. Motor symptoms include tremor, rigidity, slow movement (bradykinesia), and loss of the ability to initiate movement (akinesia) (collectively, the “off” state). Non-motor symptoms of PD include dementia, dysphagia (difficulty swallowing), slurred speech, orthostatic hypotension, seborrheic dermatitis, urinary incontinence, constipation, mood alterations, sexual dysfunction, and sleep issues (e.g., daytime somnolence, insomnia).

After more than 40 years of clinical use levodopa (LD) therapy remains the most effective method for managing PD and provides the greatest improvement in motor function. Consequently, LD administration is the primary treatment for PD. LD is usually orally administered. The orally administered LD enters the blood and part of the LD in the blood crosses the blood brain barrier. It is metabolized, in part, in the brain to dopamine which temporarily diminishes the motor symptoms of PD. As the neurodegeneration underlying PD progresses, the patients require increasing doses of LD and the fluctuations of brain dopamine levels increase. When too much LD is transported to the brain, dyskinesia sets in (uncontrolled movements such as writhing, twitching and shaking); when too little is transported, the patient re-enters the off state. Furthermore, as PD progresses, the therapeutic window for oral formulations of LD narrows, and it becomes increasingly difficult to control PD motor symptoms without inducing motor complications. In addition, most PD patients develop response fluctuations to intermittent oral LD therapy, such as end of dose wearing off, sudden on/offs, delayed time to on, and response failures.

Methods and devices that permit continuous or semi-continuous administration of drugs to subjects in order to produce reliable and steady pharmacokinetic performance are needed to improve efficacy and/or safety of therapies, particularly for drugs with a short half-life (e.g., in the plasma), and/or short persistence of the drug's physiological effect, and/or a narrow therapeutic window.

### SUMMARY OF THE INVENTION

The invention features methods for continuously administering a drug by oral infusion (e.g., via a drug delivery device anchored in the mouth for continuously administering a pharmaceutical composition), and pre-screening subjects and, if necessary, treating subjects with a Helicobacter pylori (H. pylori) infection to improve the pharmacokinetic performance of the continuous administration in the subject.

The invention features a method of administering a pharmaceutical composition to a subject, the method including: (i) providing a subject identified as being free of infection by H. pylori; (ii) inserting a drug delivery device into the mouth of the subject, the drug delivery device including a drug reservoir containing at least one drug in the pharmaceutical composition; and (iii) continuously or semicontinuously administering the pharmaceutical composition into the mouth of a subject at a rate between 0.001 mL/hour and 1.25 mL/hour.

The invention further features a method of administering a pharmaceutical composition to a subject, the method including: (i) providing a subject, wherein the subject has been tested for an infection by H. pylori, and, if infected, treated for the H. pylori infection; (ii) inserting a drug delivery device into the mouth of the subject, the drug delivery device including a drug reservoir containing at least one drug in the pharmaceutical composition; and (iii) continuously or semicontinuously administering the pharmaceutical composition into the mouth of a subject at a rate between 0.001 mL/hour and 1.25 mL/hour.

The invention also features use of levodopa or a levodopa prodrug in the treatment of Parkinson's disease, in which the levodopa or levodopa prodrug is administered via a drug delivery device, the use including: (i) providing a subject identified as being free of infection by H. pylori; (ii) inserting the drug delivery device into a mouth of the subject, the drug delivery device including a drug reservoir containing a pharmaceutical composition including the levodopa or levodopa prodrug; and (iii) continuously or semicontinuously administering the levodopa or levodopa prodrug into the mouth of the subject at a rate between 0.001 mL/hour and 1.25 mL/hour.

The invention further features use of levodopa or a levodopa prodrug in the treatment of Parkinson's disease, in which the levodopa or levodopa prodrug is administered via a drug delivery device, the use including: (i) providing a subject, wherein the subject has been tested for an infection by H. pylori, and, if infected, treated for the H. pylori infection; (ii) inserting the drug delivery device into a mouth of the subject, the drug delivery device including a drug reservoir containing a pharmaceutical composition including the levodopa or levodopa prodrug; and (iii) continuously or semicontinuously administering the levodopa or levodopa prodrug into the mouth of the subject at a rate between 0.001 mL/hour and 1.25 mL/hour.

In some embodiments, the use includes administering into the subject's mouth the levodopa or levodopa prodrug for a period of at least 4 hours at an hourly rate in the range of 30 mg/hour to 150 mg/hour. In some embodiments, a circulating plasma levodopa concentration greater than 1,200 ng/mL and less than 2,500 ng/mL is continuously maintained for a period of at least 4 hours during the administration. In some embodiments, the subject has a score of 4 and 5 on the Hoehn and Yahr scale. In some embodiments, the pharmaceutical composition further includes carbidopa or a carbidopa prodrug. In some embodiments, the pharmaceutical composition further includes benserazide.

The invention also features use of baclofen or a baclofen prodrug in the treatment of spasticity, in which the baclofen or baclofen prodrug is administered via a drug delivery device, the use including (i) providing a subject identified as being free of infection by H. pylori; (ii) inserting the drug delivery device into a mouth of the subject, the drug delivery device including a drug reservoir containing a pharmaceutical composition including the baclofen or baclofen prodrug; and (iii) continuously or semicontinuously administering the baclofen or baclofen prodrug into the mouth of the subject at a rate between 0.001 mL/hour and 1.25 mL/hour.

The invention further features use of baclofen or a baclofen prodrug in the treatment of spasticity, in which the baclofen or baclofen prodrug is administered via a drug delivery device, the use including: (i) providing a subject, wherein the subject has been tested for an infection by H. pylori, and, if infected, treated for the H. pylori infection; (ii) inserting the drug delivery device into a mouth of the subject, the drug delivery device including a drug reservoir containing a pharmaceutical composition including the baclofen or baclofen prodrug; and (iii) continuously or semicontinuously administering the baclofen or baclofen prodrug into the mouth of the subject at a rate between 0.001 mL/hour and 1.25 mL/hour.

In some embodiments, the subject to be treated with baclofen or a baclofen prodrug has multiple sclerosis or a spinal cord injury.

The invention also features use of pyridostigmine or a pyridostigmine prodrug in the treatment of myasthenia gravis, in which the pyridostigmine or pyridostigmine prodrug is administered via a drug delivery device, the use including: (i) providing a subject identified as being free of infection by H. pylori; (ii) inserting the drug delivery device into a mouth of the subject, the drug delivery device including a drug reservoir containing a pharmaceutical composition including the pyridostigmine or pyridostigmine prodrug; and (iii) continuously or semicontinuously administering the pyridostigmine or pyridostigmine prodrug into the mouth of the subject at a rate between 0.001 mL/hour and 1.25 mL/hour.

The invention further features use of pyridostigmine or a pyridostigmine prodrug in the treatment of myasthenia gravis, in which the pyridostigmine or pyridostigmine prodrug is administered via a drug delivery device, the use including: (i) providing a subject, wherein the subject has been tested for an infection by H. pylori, and, if infected, treated for the H. pylori infection; (ii) inserting the drug delivery device into a mouth of the subject, the drug delivery device including a drug reservoir containing a pharmaceutical composition including the pyridostigmine or pyridostigmine prodrug; and (iii) continuously or semicontinuously administering the pyridostigmine or pyridostigmine prodrug into the mouth of the subject at a rate between 0.001 mL/hour and 1.25 mL/hour.

The invention also features use of pilocarpine or a pilocarpine prodrug in the treatment of dry mouth, in which the pilocarpine or pilocarpine prodrug is administered via a drug delivery device, the use including: (i) providing a subject identified as being free of infection by H. pylori; (ii) inserting the drug delivery device into a mouth of the subject, the drug delivery device including a drug reservoir containing a pharmaceutical composition including the pilocarpine or pilocarpine prodrug; and (iii) continuously or semicontinuously administering the pilocarpine or pilocarpine prodrug into the mouth of the subject at a rate between 0.001 mL/hour and 1.25 mL/hour.

The invention further features se of pilocarpine a pilocarpine prodrug in the treatment of dry mouth, in which the pilocarpine or pilocarpine prodrug is administered via a drug delivery device, the use including: (i) providing a subject, wherein the subject has been tested for an infection by H. pylori, and, if infected, treated for the H. pylori infection; (ii) inserting the drug delivery device into a mouth of the subject, the drug delivery device including a drug reservoir containing a pharmaceutical composition including the pilocarpine or pilocarpine prodrug; and (iii) continuously or semicontinuously administering the pilocarpine or pilocarpine prodrug into the mouth of the subject at a rate between 0.001 mL/hour and 1.25 mL/hour.

In some embodiments, the dry mouth is associated with Sjogren's syndrome or radiation therapy.

The invention also features use of furosemide or a furosemide prodrug in the treatment of high blood pressure (hypertension) or edema, in which the furosemide or furosemide prodrug is administered via a drug delivery device, the use including: (i) providing a subject identified as being free of infection by H. pylori; (ii) inserting the drug delivery device into a mouth of the subject, the drug delivery device including a drug reservoir containing a pharmaceutical composition including the furosemide or furosemide prodrug; and (iii) continuously or semicontinuously administering the furosemide or furosemide prodrug into the mouth of the subject at a rate between 0.001 mL/hour and 1.25 mL/hour.

The invention further features use of furosemide or a furosemide prodrug in the treatment of high blood pressure (hypertension) or edema, in which the furosemide or furosemide prodrug is administered via a drug delivery device, the use including: (i) providing a subject, wherein the subject has been tested for an infection by H. pylori, and, if infected, treated for the H. pylori infection; (ii) inserting the drug delivery device into a mouth of the subject, the drug delivery device including a drug reservoir containing a pharmaceutical composition including the furosemide or furosemide prodrug; and (iii) continuously or semicontinuously administering the furosemide or furosemide prodrug into the mouth of the subject at a rate between 0.001 mL/hour and 1.25 mL/hour.

In some embodiments, the edema is associated with heart failure (e.g., congestive heart failure), kidney disease, or liver disease (e.g., cirrhosis or liver fibrosis).

The drug delivery device can be removably attached to an intraoral surface of the subject. In some embodiments, the pharmaceutical composition is administered to the subject over a delivery period of 4, 8, 16, 24, or more hours. In some embodiments, the rate of delivery of the pharmaceutical composition during each hour of the delivery period is within +/−25% or +/−10% of the average rate of delivery during the delivery period. In particular embodiments, the half-life of the drug is less than 8 hours, 6 hours, 4 hours, 2 hours, 1 hour, 30 min, 20 min or 10 min. In particular embodiments, the fluctuation index of the drug is less than or equal to 2.0, 1.5, 1.0, 0.75, 0.50, 0.25, or 0.15 during the delivery period. In certain embodiments, the fluctuation index of the drug is less than the average fluctuation index observed for subjects of the same condition and receiving the same therapy but having an H. pylori infection.

The method or use can include administering the drug from a drug delivery device to the subject for a delivery period of not less than about 4 hours and not more than about 7 days. In some embodiments, the drug reservoir includes a volume of a pharmaceutical composition, and the method or use further includes oral administration at a rate in the range of from 15 μL per hour to about 1.25 mL per hour during the delivery period. For example, the method or use can include oral administration at a rate in the range of from about 0.015 mL/hour to about 0.25 mL/hour; from about 0.25 mL/hour to about 0.5 mL/hour; from about 0.5 mL/hour to about 0.75 mL/hour; or from about 0.75 mL/hour to about 1.0 mL/hour.

In certain embodiments, the drug is administered to the subject at an average rate of not less than 0.01 mg per hour and not more than 250 mg per hour. For example, the drug can be administered to the subject at an hourly rate in the range of 0.01 mg per hour to 1 mg per hour, 1 mg per hour to 10 mg per hour, 10 mg per hour to 100 mg per hour, or 100 mg per hour to 250 mg per hour.

In some embodiments, the pharmaceutical composition is administered to the subject at least once every 60 minutes. For example, the pharmaceutical composition can be administered to the subject at least once every 30 minutes; at least once every 15 minutes, or, desirably, the pharmaceutical composition is administered to the subject continuously.

In particular embodiments, the method further includes treating a disease in the subject, in which the disease is mucositis, allergy, an immune disease, anesthesia, a bacterial infection, a viral infection, a fungal infection, a parasite-associated disorder, a sterile inflammatory disease, cancer, pain, organ transplantation, disordered sleep, epilepsy or a seizure, anxiety, a mood disorder, post-traumatic stress disorder, arrhythmia, hypertension, heart failure, spasticity, or diabetic nephropathy.

In some embodiments, the method further includes treating a disease in the subject, wherein the disease is multiple sclerosis, cerebral palsy, spasticity, neurogenic orthostatic hypotension, Wilson's disease, cystinuria, rheumatoid arthritis, Alzheimer's disease, Type-1 Gaucher disease, Type C Niemann-Pick disease, eosinophilic gastroenteritis, chronic mastocytosis, ulcerative colitis, gastro-oesophageal reflux, gastroenteritis, hyperemesis gravidarum, glioblastoma multiformae, anaplastic astrocytoma, pulmonary hypertension, coronary heart disease congestive heart failure, angina, Type 2 diabetes, COPD, asthma, irritable bowel syndrome, overactive bladder, or urinary urge incontinence.

In some embodiments, the pharmaceutical composition includes one or more drugs selected from methylphenidate, levodopa or a levodopa prodrug, baclofen or a baclofen prodrug, pyridostigmine or a pyridostigmine prodrug, pilocarpine or a pilocarpine prodrug, furosemide or a furosemide prodrug, a prostaglandin, prostacyclin, treprostinil, beraprost, nimodipine, and testosterone, or any other drug described herein.

In particular embodiments of the method: (a) the subject has Parkinson's disease, (b) the drug reservoir includes levodopa or a levodopa prodrug, and (c) the method includes administering into the subject's mouth the levodopa or a levodopa prodrug for a period of at least 4 hours at an hourly rate in the range of 30 mg/hour to 150 mg/hour. The method can include intraoral administration such that a circulating plasma levodopa concentration greater than 1,200 ng/mL and less than 2,500 ng/mL is continuously maintained for a period of at least 4 hours during the administration. In certain embodiments, the subject has a score of 4 and 5 on the Hoehn and Yahr scale.

In certain embodiments, the method includes treating spasticity in a subject in need thereof and the pharmaceutical composition includes baclofen or a baclofen prodrug.

In certain embodiments, the method includes treating myasthenia gravis in a subject in need thereof, and the pharmaceutical composition includes pyridostigmine or a pyridostigmine prodrug.

In certain embodiments, the method includes treating dry mouth in a subject in need thereof and the pharmaceutical composition includes pilocarpine or a pilocarpine prodrug. In some embodiments, the dry mouth is associated with or caused by Sjogren's syndrome or radiation therapy.

In certain embodiments, the method includes treating high blood pressure (hypertension) or edema in a subject in need thereof, and the pharmaceutical composition includes furosemide or a furosemide prodrug. In some embodiments, the edema is associated with or caused by heart failure (e.g., congestive heart failure), kidney disease, or liver disease (e.g., cirrhosis or liver fibrosis).

In one embodiment of any of the above methods or uses, during the administration the circulating drug plasma concentration varies by less than +/−20% or +/−10% from its mean for a period of at least 1, 2, or 4 hours.

In another embodiment of any of the above methods or uses, an efficacious circulating plasma concentration of the drug is continuously maintained for a period of at least 8 hours during the administration.

The drug delivery device used in the methods or uses of the invention can be configured to be removably inserted in a subject's mouth and for continuous or semi-continuous intraoral administration of the drug, the device including: (i) a fastener to removably secure the drug delivery device to a surface of the subject's mouth; (ii) an electrical or mechanical pump, the latter exemplified by a propellant-driven deformable diaphragm-comprising pump; and (iii) a drug reservoir containing the pharmaceutical composition, the volume of the drug reservoir being from 0.1 mL to 5 mL.

The methods or uses of the invention can be performed with a drug delivery device that can removably secure to one or more teeth of the patient. In some embodiments, the fastener that removably secures the drug delivery device to one or more teeth includes a band, a bracket, a clasp, a splint, or a retainer. For example, the fastener may include a transparent retainer or a partial retainer attachable to fewer than 5 teeth.

The methods or uses of the invention can include removably attaching a drug delivery device of the invention to an intraoral surface of the patient. In certain embodiments, the method or use further includes detaching the device from the intraoral surface and/or administering drug to the patient for a delivery period of not less than about 4 hours and not more than about 7 days. In some embodiments, the drug delivery device includes a drug reservoir including a volume of a drug, and the method or use further includes oral administration at a rate in the range of from 15 μL per hour to about 1.25 mL per hour (e.g., as described herein) during the delivery period. In particular embodiments, the fluctuation index of the drug is less than or equal to 2.0, 1.5, 1.0, 0.75, 0.50, 0.25, or 0.15 during the delivery period. In some embodiments, the drug delivery device can include a drug reservoir including a pharmaceutical composition including a drug and the drug is administered to the patient at an average rate of not less than 0.01 mg per hour and not more than 125 mg per hour (e.g., from about 0.01 mg/hour to about 125 mg/hour, from about 0.05 mg/hour to about 125 mg/hour, from about 0.10 mg/hour to about 125 mg/hour, from about 0.50 mg/hour to about 125 mg/hour, from about 1.0 mg/hour to about 125 mg/hour from about 5.0 mg/hour to about 125 mg/hour, from about 10 mg/hour to about 125 mg/hour, from about 25 mg/hour to about 125 mg/hour, from about 50 mg/hour to about 125 mg/hour, from about 100 mg/hour to about 125 mg/hour, from about 0.01 mg/hour to about 100 mg/hour, from about 0.01 mg/hour to about 50 mg/hour, from about 0.01 mg/hour to about 25 mg/hour, from about 0.01 mg/hour to about 10 mg/hour, from about 0.01 mg/hour to about 5.0 mg/hour, from about 0.01 mg/hour to about 1.0 mg/hour, from about 0.01 mg/hour to about 0.5 mg/hour, from about 0.01 mg/hour to about 0.25 mg/hour, from about 0.01 mg/hour to about 0.1 mg/hour, from about 0.01 mg/hour to about 0.05 mg/hour, or from about 1 mg/hour to about 10 mg/hour, from about 10 mg/hour to about 100 mg/hour). In some embodiments, the pharmaceutical composition can be administered to the patient at least once every 60 minutes, at least once every 30 minutes, or at least once every 15 minutes. In other embodiments, the pharmaceutical composition is administered to the patient continuously. In certain embodiments, the pharmaceutical composition can be administered to the patient over a delivery period of about 8 or more hours (e.g., more than 10, 12, 14, 16, 18, 20, or 24 hours).

In certain embodiments, a method of administering a pharmaceutical composition of the invention further includes treating a disease in the patient, wherein the disease is spasticity, muscle weakness, mucositis, allergy, immune disease, anesthesia, a bacterial infection, a viral infection, a fungal infection, a sterile inflammatory disease, cancer, pain, organ transplantation, disordered sleep, epilepsy or a seizure, anxiety, a mood disorder, post-traumatic stress disorder, arrhythmia, hypertension, heart failure, or diabetic nephropathy.

In one particular embodiment of any of the above methods, the method further includes treating a disease in the patient, wherein the disease is multiple sclerosis, cerebral palsy, spasticity, neurogenic orthostatic hypotension, Wilson's disease, cystinuria, rheumatoid arthritis, Alzheimer's disease, myasthenia gravis, Type-1 Gaucher disease, Type C Niemann-Pick disease, eosinophilic gastroenteritis, chronic mastocytosis, ulcerative colitis, gastro-oesophageal reflux, gastroenteritis, hyperemesis gravidarum, glioblastoma multiformae, anaplastic astrocytoma, pulmonary hypertension, coronary heart disease, congestive heart failure, angina, Type 2 diabetes, COPD, asthma, irritable bowel syndrome, overactive bladder, and urinary urge incontinence. In one particular embodiment, the method includes treating myasthenia gravis and the pharmaceutical composition includes pyridostigmine, or a pharmaceutically acceptable salt thereof.

In one particular embodiment, the pharmaceutical composition includes one or more drugs selected from levodopa or a levodopa prodrug, baclofen or a baclofen prodrug, pyridostigmine or a pyridostigmine prodrug, pilocarpine or a pilocarpine prodrug, furosemide or a furosemide prodrug, methylphenidate, prostaglandins, prostacyclin, treprostinil, beraprost, nimodipine, and testosterone. In still other embodiments, the pharmaceutical composition includes a mucoadhesive polymer.

In any of the methods of the invention, the pharmaceutical composition may include one or more of levodopa or a levodopa prodrug, baclofen or a baclofen prodrug, pyridostigmine or a pyridostigmine prodrug, pilocarpine or a pilocarpine prodrug, furosemide or a furosemide prodrug, methylphenidate, prostaglandins, prostacyclin, treprostinil, beraprost, nimodipine, and testosterone.

In any of the methods or uses of the invention, the pharmaceutical composition may include drug in the form of particles.

### Abbreviations and Definitions

The term “about,” as used herein, refers to a number that is ±10% of a value that this term precedes except when the value is that of a temperature. For temperatures “about” means ±3° C.

The term “administration” or “administering” refers to a method of giving a dosage of a therapeutic drug, such as LD and/or carbidopa (CD), to a patient. The drug may be formulated as a fluid, such as a viscous suspension, such as a paste. Fluids may be infused. The dosage form of the invention is preferably administered into the mouth, optionally using a drug delivery device such as an infusion pump. The drug can be swallowed and/or absorbed anywhere within the mouth or via the stomach. Typical durations of administration from a single device or drug reservoir are greater than 4, 8, 12, or 16 hours per day, up to and including 24 hours per day. Administration can also take place over multiple days from a single device or drug reservoir, e.g., administration of a drug for 2 or more days, 4 or more days, or 7 or more days.

The term “CD” refers to Carbidopa.

The term “COMT” refers to catechol-O-methyl transferase.

As used herein “continuous administration” or “continuous infusion” refers to uninterrupted administration or infusion of a drug in solid or fluid form.

As used herein, the term “fastener” refers to an element for attaching the device of the invention, or its components, to a surface of the mouth (e.g., to the teeth). Exemplary methods of attachment are fasteners banded, adhered, cemented or glued to one, two or more teeth; dental appliances; splints; transparent retainers; metal wire Hawley retainers; partial retainers on one side of the mouth (e.g., attached to 3, 4, or 5 teeth); thermo or vacuum-formed Essix retainers typically including a polypropylene or polyvinylchloride material, typically 0.020″ or 0.030″ thick; thermo-formed Zendura retainers including polyurethane; bonded (fixed) retainers including a passive wire bonded to the tongue-side of lower or upper teeth; muco-adhesives that adhere to the oral mucosal tissue and slowly erode; and fasteners that conform or are molded to fit a patient's teeth or soft tissue, similar to dental splints used to treat bruxism and sleep apnea. Similarly, the drug delivery devices, drug pumps, drug reservoirs and other devices of the invention may be directly or indirectly attached to a removable denture, a prosthetic tooth crown, a dental bridge, a moral band, a bracket, a mouth guard, or a dental implant.

As used herein the term “fluctuation index” refers to the magnitude of the rise and fall of drug level in plasma relative to the average plasma concentration, and is defined as [Cₘₐₓ−Cₘᵢₙ]/C_{avg}. The fluctuation index is measured over a specified period of time. The time period can begin, for example, after the drug's plasma concentration: has reached the steady-state concentration; or has reached 90% of the steady-state concentration; or 30, 60, or 120 minutes after any of the drug delivery devices of the invention has been inserted into the mouth and begun to deliver drug. The time period can end, for example: at the end of the use period specified in the instructions for use of the drug delivery device; when the drug reservoir is 90% depleted or substantially depleted; or about 4, 8, 16, 24, 72, or 168 hours after the start of the time period.

As used herein, the term “fluid” encompasses any drug-including liquid or gel that can be pumped, or any non-pourable suspension, such as paste, that can be extruded. The fluid can be a

Newtonian or a non-Newtonian fluid; it can be an easy to deform solid or a soft paste, which may move as a plug via slip flow. It can be, for example, a viscous Newtonian or non-Newtonian suspension. The term encompasses, for example, true solutions, colloidal solutions, emulsions, pastes, suspensions, and dense semi-solid toothpaste-like suspensions deforming under pressure sufficiently to be extruded into the mouth. The fluid infused can be aqueous, non-aqueous, single phase, two-phase, three- phase or multiphase. The emulsions can be, for example, oil-in-water or water-in-oil, and can include micelles and/or liposomes.

As used herein, H. pylori refers to Helicobacter pylori.

As used herein, “infused” or “infusion” includes infusion into the mouth.

The term “LD” refers to levodopa, also known as L-DOPA, or a salt thereof.

As used herein, “mechanical pump” means any drug delivery device whose motive force is not electricity, magnetism, or gravity. Examples of mechanical pumps include drug delivery devices wherein the drug is delivered by the force or pressure of a spring, an elastomer, a compressed gas, or a propellant.

As used herein, “mouth” includes the areas of the oral cavity, including those areas of the oral cavity adjacent the lips, cheeks, gums, teeth, tongue, roof of the mouth, hard palate, soft palate, tonsils, uvula, and glands.

The abbreviation “M” means moles per liter. Usage of the term does not imply, as it often does in chemistry, that the drug is dissolved. As used herein 1 M means that a 1 liter volume contains 1 mole of the combination of the undissolved (often solid) and/or the dissolved drug. For example, 1 M LD means that there is 197 mg of solid (undissolved) and dissolved LD in one mL.

The term “PD” refers to Parkinson's disease, including patients with scores of 4 and 5 on the Hoehn and Yahr scale.

The term “prodrug,” as used herein, represents compounds that are rapidly transformed in vivo to the parent compound, for example, by hydrolysis in blood. Prodrugs may be conventional esters. Some common esters that have been utilized as prodrugs are phenyl esters, aliphatic (C8-024) esters, acyloxymethyl esters, carbamates, and amino acid esters. For example, a compound that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26: 4351-4367, 1996, each of which is incorporated herein by reference.

As used herein, “pump” refers to any mechanism capable of administering a fluid formulated drug product over a period of 4 or more hours. Examples of pumps include battery-powered pumps (e.g., syringe pumps, piezoelectric, peristaltic pumps, or diaphragm pumps), mechanical devices with or without moving parts that are not battery-powered (e.g., liquefied propellant driven pumps, gas-driven pumps, spring-driven pumps, shape memory alloy driven pumps, and elastomeric pumps), osmotic pumps, and battery-operated electroosmotic pumps (with or without moving parts).

The terms “semi-continuous administration” and “frequent administration,” as used interchangeably herein, refer to an administration (e.g., infusion) of a drug in solid or fluid form at a frequency of at least once every 120 minutes, and preferably at least every 90, 60, 30, 15, or 5 minutes.

As used herein, the term “suitable for continuous or frequent intermittent intra-oral delivery” refers to drug particle suspensions of the invention that are efficacious and safe upon intra-oral delivery. For example, local adverse events in or near the mouth (if any) produced by continuous or frequent intermittent intra-oral administration of the suspension are tolerable or mild.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to ameliorate the disease and improve the patient's condition. The term “treating” also includes treating a patient to delay progression of a disease or its symptoms. Thus, in the claims and embodiments, treating is the administration to a patient either for therapeutic or prophylactic purposes.

As used herein “viscosity” means dynamic viscosity also known as shear viscosity, typically measured at a low shear rate.

Other features and advantages of the invention will be apparent from the following Detailed Description and the claims.

### DETAILED DESCRIPTION

The invention features methods for continuously administering a drug by oral infusion (e.g., via a drug delivery device anchored in the mouth for continuously administering a pharmaceutical composition), and pre-screening subjects and, if necessary, treating subjects with H. pylori to improve the pharmacokinetic performance of the continuous administration in the subject.

Using the methods of the invention, drugs can be administered intraorally (i.e., onto or near any intraoral surface, e.g., the lips, cheeks, gums, teeth, tongue, roof of the mouth, hard palate, soft palate, tonsils, uvula, and glands). The drugs administered intraorally are typically swallowed by the patient, together with the patient's saliva. The drugs can be diluted by the patient's saliva and can optionally be partly or fully dissolved in the saliva. The drugs can be absorbed in the patient's gastrointestinal tract, e.g., in the small intestines or large intestines. The methods of the invention permit continuous or semi-continuous administration of drugs to subjects in order to produce reliable and steady pharmacokinetic performance are needed to improve efficacy and/or safety of therapies, particularly for drugs with a short half-life (e.g., in the plasma), and/or short persistence of the drug's physiological effect, and/or a narrow therapeutic window.

### H. pylori Testing and Treatment

Most people don't realize they have H. pylori infection because they rarely get sick from it. H. pylori infection occurs among 48.5% of the general population worldwide, with high geographic variability (see, e.g., Hooi et al., Gastroenterology. 153: 420 (2017)). Diagnostic approaches for H. pylori infection include invasive and non-invasive testing (see, e.g., Zagari et al., Dig Liver Dis. 47: 903 (2015); and Fallone et al., Gastroenterology. 151: 51 (2016)). The 13 C-urea breath test is established as a highly sensitive and specific test (96% sensitivity and 93% specificity) for the non-invasive diagnosis of infection (see, e.g., Ferwana et al., World J Gastroenterol. 21: 1305 (2015)). A stool-based monoclonal antigen test is also available (see, e.g., Gisbert et al., Am J Gastroenterol. 101: 1921 (2006)).

In accordance with the methods of the invention, subjects identified as being H. pylori positive can be the treated by any recommended eradication therapy, exemplified by treatment with the combination of oral esomeprazole 40 mg daily, oral clarithromycin 500 mg twice daily, and oral amoxicillin 1000 mg twice daily for 1 week. Other therapeutic treatments can also be used to eradicate the H. pylori infection.

For subjects receiving continuous or semi-continuous intraoral infusion therapy, it can be advantageous for the subjects to be H. pylori negative in order to reduce the fluctuation index of the drug being intraorally infused.

### Therapy

The methods of the invention can be used in conjunction with the administration of a variety of drugs that have a short half-life and/or a narrow therapeutic range. Complementary drugs may be co-administered or co-infused with these drugs. Such complementary drugs may improve the pharmacokinetics, improve the efficacy, and/or reduce the side effects of the primary drugs.

The conversion of LD to 3-OMD is catalyzed by the enzyme catechol-O-methyl transferase, COMT. Administration of a COMT inhibitor can reduce the required dosage of LD or LD/CD, or in earlier stages of PD, even provide for managing the disease without LD or LD/CD. The two most frequently used COMT inhibitors, entacapone and tolcapone are, however short-lived. Entacapone does not cross the blood-brain barrier and can be less toxic than Tolcapone, which crosses the barrier. The plasma half-life of Entacapone is, however, merely 0.4-0.7 hours, making it difficult to maintain a sufficient plasma level of the drug without administering large and frequent doses of the drug. In clinical practice, one 200 mg tablet is often administered with each LD/CD or LD/Benserazide dose. The maximum recommended dose is 200 mg ten times daily, i.e., 2,000 mg. Continuous oral administration of Entacapone could reduce the dosage and/or frequency of administration of the drug and its side effects. The reduced dosage could alleviate side effects such as dyskinesia and/or gastrointestinal problems, nausea, abdominal pain or diarrhea. Entacapone could be continuously orally administered in a daily dose of less than 1000 mg per 16 hours while the patient is awake (such as less than 500 mg per 16 awake hours), for example as an aqueous suspension including small particles, e.g., less than 100 μm average diameter, such as less than 30 μm, 10 μm, 3 μm or 1 μm particles of Entacapone. Alternatively, it could be administered as a suspension in a non-aqueous solution, such an edible oil, cocoa-butter, propylene glycol, or glycerol. Tolcapone is a reversible COMT inhibitor of 2-3 hour half-life. It exerts its COMT inhibitory effects in the central nervous system as well as in the periphery. Its use is limited by its hepatotoxicity. The typical dose of Tolcapone in PD management is 100-200 mg three times daily. Tolcapone may also be effective in the treatment of Hallucinogen Persisting Perception Disorder, decreasing visual symptoms. Continuous oral administration of Tolcapone could reduce its dosage and/or frequency of administration and its hepatotoxicity. The reduced dosage could alleviate its hepatotoxicity. Its daily dose could be less than 500 mg per 16 awake hours, such as less than 300 mg per 16 awake hours. It could be continuously orally administered, for example, as a suspension including small particles, e.g., less than 100 μm average diameter, such as less than 30 μm, 10 μm, 3 μm or 1 μm particles of the drug.

Because administration is into the mouth, it is preferred that the drugs selected for administration are those whose taste is neutral or pleasant, as perceived by a majority of patients. Taste masking or modifying excipients may be added to the formulations of drugs whose taste is unpleasant, as perceived by a majority of patients.

Other drugs that may usefully be delivered in accordance with the invention include methylphenidate, prostaglandins, prostacyclin, treprostinil, beraprost, nimodipine, pyridostigmine or a pyridostigmine prodrug, pilocarpine or a pilocarpine prodrug, levodopa or a levodopa prodrug, baclofen or a baclofen prodrug, furosemide or a furosemide prodrug, and testosterone. Levodopa prodrug formulations of are provided in U.S. Pat. No. 5,607,969, and in patent applications WO 2012/079072 and WO 2013/184646, each incorporated herein by reference. The preferred LD prodrugs for administration into the mouth include highly soluble levodopa amides, levodopa esters, levodopa carboxamides, levodopa sulfonamide, levodopa phosphate prodrugs (e.g., foslevodopa, also known as levodopa 4′-monophosphate, see, e.g., Huters et al., J Org. Chem. 2021 and Rosebraugh et al., Ann Neurol. 90: 52-61, 2021), levodopa ethyl ester, levodopa methyl ester, and their salts. An exemplary baclofen prodrug is arbaclofen placarbil. Exemplary pilocarpine prodrugs include alkyl and aralkyl esters of pilocarpic acid, pilocarpic acid diesters, and other pilocarpic acid derivatives, such as those described in Bundgaard et al., J Pharm Sci 75: 36-43, 1986, Bundgaard et al., J Pharm Sci 75:775-83, 1986, and U.S. Pat. No. 4,742,073A, which are incorporated herein by reference. Exemplary furosemide prodrugs include furosemide esters, such as the neutral alkyl ester, alkyl esters containing an amino group, glycolamide esters and 0-acyloxymethyl ester described in Mork et al., International Journal of Pharmaceutics 60: 163-169, 1990, acyloxymethyl esters of furosemide described in Prandi et al., Farmaco 47: 249-63, 1992 and Prandi et al., Farmaco 47: 1225-34, 1992, and the furosemide analogs and prodrugs described in International Patent Application Publication No. WO2014039454A2, which are incorporated herein by reference.

Examples of drugs that are often prescribed to be dosed four times per day include:
Amoxicillin—infection
Cephalexin (Keflex)—infection
Chlorpromazine (Thorazine)—neuroleptic for migraine
Diazepam (Valium)—anxiety and sleep
Diclofenac (Voltaren)—arthritis
Diltiazem—calcium channel blocker
Erythromycin—infection
Haloperidol (Haldol)—neuroleptic for migraine
Impramine—psychotropic
Ipratropium (Atrovent)—Anticholinergic
Metoclopramide (Reglan)—gastroesophageal reflux, migraine
Nifedipine—calcium channel blocker
Olanzapine (Zyprexa)—neuroleptic for migraine
Prochlorperazine (Compazine)—neuroleptic for migraine
Promethazine (Phenergan)—neuroleptic for migraine
Salbutamol—asthma
Tetracycline—infection
Theophylline (Theolair)—COPD, asthma
Trazodone—psychotropic
Vaccines—immunotherapy
Zaleplon—disordered sleep, insomnia

Drugs delivered as solids may be formulated with excipients to increase disintegration or dispersion.

Many types of drugs may be delivered in accordance with the invention. Drugs which may in principle be used for treatment according to the invention are any known drugs, wherein the drugs may be present in the form according to the invention as such, or in the form of the active ingredient, optionally in the form of a pharmaceutically acceptable salt of the active ingredient. Drugs which may be delivered in accordance with the invention include, without limitation, analgesics and anti-inflammatory agents (e.g., aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac), antihelmintics (e.g., albendazole, bephenium hydroxynaphthoate, cambendazole, dichlorophen, ivermectin, mebendazole, oxamniquine, oxfendazole, oxantel embonate, praziquantel, pyrantel embonate, thiabendazole), anti-arrhythmic agents (e.g., amiodarone HCl, disopyramide, flecainide acetate, quinidine sulphate, anti-bacterial agents (e.g., benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim), anti-coagulants (e.g., dicoumarol, dipyridamole, nicoumalone, phenindione), antidepressants (e.g., amoxapine, maprotiline HCl, mianserin HCl, nortriptyline HCl, trazodone HCl, trimipramine maleate), antidiabetics (e.g., acetohexamide, chlorpropamide, glibenclamide, gliclazide, glipizide, tolazamide, tolbutamide), anti-epileptics (e.g., beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid, topiramate, lamotrigine, gabapentin, levetiracetam, pregabalin), antifungal agents (e.g., amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid), antigout agents (e.g., allopurinol, probenecid, sulphin-pyrazone), antihypertensive agents (e.g., amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCl, reserpine, terazosin HCl), antimalarials (e.g., amodiaquine, chloroquine, chlorproguanil HCl, halofantrine HCl, mefloquine HCl, proguanil HCl, pyrimethamine, quinine sulphate), anti-migraine agents (e.g., dihydroergotamine mesylate, ergotamine tartrate, methysergide maleate, pizotifen maleate, sumatriptan succinate), anti-muscarinic agents (e.g., atropine, benzhexol HCl, biperiden, ethopropazine HCl, hyoscyamine, mepenzolate bromide, oxyphencyclimine HCl, tropicamide), anti-neoplastic agents and immunosuppressants (e.g., aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone), anti-protazoal agents (e.g., benznidazole, clioquinol, decoquinate, diiodohydroxyquinoline, diloxanide furoate, dinitolmide, furzolidone, metronidazole, nimorazole, nitrofurazone, ornidazole, tinidazole), anti-thyroid agents (e.g., carbimazole, propylthiouracil), anxiolytic, sedatives, hypnotics and neuroleptics (e.g., alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone), β-Blockers (e.g., acebutolol, alprenolol, atenolol, labetalol, metoprolol, nadolol, oxprenolol, pindolol, propranolol), cardiac inotropic agents (e.g., amrinone, digitoxin, digoxin, enoximone, lanatoside C, medigoxin), corticosteroids (e.g., beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone), diuretics (e.g., acetazolamide, amiloride, bendrofluazide, bumetanide, chlorothiazide, chlorthalidone, ethacrynic acid, furosemide (frusemide), metolazone, spironolactone, triamterene), anti-parkinsonian agents (e.g., bromocriptine mesylate, lysuride maleate), gastrointestinal agents (e.g., bisacodyl, cimetidine, cisapride, diphenoxylate HCl, domperidone, famotidine, loperamide, mesalazine, nizatidine, omeprazole, ondansetron HCl, ranitidine HCl, sulphasalazine), histamine H,-receptor antagonists (e.g., acrivastine, astemizole, cinnarizine, cyclizine, cyproheptadine HCl, dimenhydrinate, flunarizine HCl, loratadine, meclozine HCl, oxatomide, terfenadine), lipid regulating agents (e.g., bezafibrate, clofibrate, fenofibrate, gemfibrozil, probucol), nitrates and other anti-anginal agents (e.g., amyl nitrate, glyceryl trinitrate, isosorbide dinitrate, isosorbide mononitrate, pentaerythritol tetranitrate), opioid analgesics (e.g., codeine, dextropropoxyphene, diamorphine, dihydrocodeine, meptazinol, methadone, morphine, nalbuphine, pentazocine), sex hormones (e.g., clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone), and stimulants (e.g., amphetamine, dexamphetamine, dexfenfluramine, fenfluramine, mazindol).

The above-stated compounds are predominantly stated by their international nonproprietary name (INN) and are known to the person skilled in the art. Further details may be found, for example, by referring to International Nonproprietary Names (INN) for Pharmaceutical Substances, World Health Organization (WHO).

The drug delivery devices can be used to orally administer drugs to patients in therapeutically effective amounts. For example, an amount is administered which prevents, delays, reduces, or eliminates the symptoms of a disease, such as PD, mucositis, bacterial infections, cancer, pain, organ transplantation, disordered sleep, epilepsy and seizures, anxiety, mood disorders, post-traumatic stress disorder, cancer, arrhythmia, hypertension, heart failure, spasticity, diabetic nephropathy, and allergy. They can also be used to manage allergies, e.g. by delivering agents used for sublingual immunotherapy such that the delivered agents contact a mucous membrane or tissue of the mouth. Using the drug delivery devices, a drug appropriate for the treatment of a given disease to be treated can be formulated and administered using the methods, compositions, and devices described herein.

Many drugs with narrow therapeutic indices benefit from drug delivery devices and methods that result in small fluctuation indices. For example, Table 1 summarizes the fluctuation indices of extended release tablet formulations of anti-epileptic drugs reported in various studies (from “Extended-release antiepileptic drugs: A comparison of pharmacokinetic parameters relative to original immediate-release formulations”, Ilo E. Leppik and Collin A. Hovinga, Epilepsia, 54(1): 28-35, 2013).

TABLE 1
Fluctuation indices of anti-epileptic drug extended release tablets.

| Drug | Fluctuation Index (SD) |
|---|---|
| Carbamazepine | 0.31 (0.1) |
| | 0.26 (0.1) |
| | 0.47 |
| | 0.49 |
| Divalproate | 0.39 (0.15) |
| sodium | 0.67 (0.16) |
| | 0.34 (0.15) |
| | 0.67 (0.17) |
| | 0.59 (0.27) |
| | 0.46 (0.16) |
| | 0.71 (0.20) |
| Lamotrigine | 0.341 |
| | 0.817 |
| | 0.209 |
| | 0.545 |
| | 0.986 |
| | 0.318 |
| Oxcarbazepine | 0.39 (0.08) |
| | 0.54 (0.09) |
| Levetiracetam | 1.19 |
| | 1.27 |

The methods of the invention can further include treating a disease or medical condition using any of the devices, drugs, formulations, and methods disclosed herein, wherein a higher fluctuation index with pills is reduced, such as to less than or equal to 2.0, 1.5, 1.0, 0.75, 0.50, 0.25, or 0.15. For example, the disease or medical condition to be treated may be Parkinson's disease, bacterial infections, cancer, pain, organ transplantation, disordered sleep, epilepsy and seizures, anxiety, mood disorders, post-traumatic stress disorder, cancer, arrhythmia, hypertension, heart failure, spasticity, dementia, diabetic nephropathy, xerostomia, and dementia.

Drug dosages administered using the methods of the invention may be higher or lower than those administered using traditional, infrequent dosing regimens. A lower daily dose is possible without loss of efficacy when continuous or semi-continuous administration reduces troughs in the drug's steady state circulating plasma concentration, enabling the drug's plasma concentration to remain above the minimum effective plasma concentration without the need for high peak concentrations. A higher daily dose is possible without increased side effects when continuous or semi-continuous administration reduces peaks in the drug's steady state circulating plasma concentration, enabling an increase in the drug's average plasma concentration without the need for high peak concentrations.

The methods of the invention provide a dosing regimen having an improved safety profile as adverse events associated with peak plasma concentrations (i.e., a Cₘₐₓ characteristic of oral unit dosage forms) are eliminated. Thus, the methods of the invention can be used to deliver drugs having a narrow therapeutic window in the patient population being treated (i.e., patients refractory to standard therapeutic regimens). Details provided below for the treatment of PD can be applicable to the formulation and administration of drugs for the treatment of other diseases.

For the treatment of PD, typical administered dose ranges are from about 20 μmole/kg to about 200 μmole/kg of LD or LD prodrug per day. The typical daily dose of the optionally co-administered DDC inhibitor is between about 5 μmole/kg and about 50 μmole/kg. For example, the typical daily dose for a patient weighing 75 kg is from about 1.5 millimoles to about 15 millimoles of LD or LD prodrug. Optionally, a molar amount of a DDC inhibitor between about 10% and about 40% of the molar amount of the LD or LD prodrug, for example between 15% and 30%, may be added. Exemplary LD prodrug formulations of the prior art are provided in U.S. Pat. No. 5,607,969, and in patent applications WO 2012/079072 and WO 2013/184646, each incorporated herein by reference. The preferred prodrugs for administration into the mouth include highly soluble levodopa amides, levodopa esters, levodopa carboxamides, levodopa sulfonamide, levodopa phosphate prodrugs (e.g., foslevodopa, also known as levodopa 4′-monophosphate, see, e.g., Huters et al., J Org. Chem. 2021 and Rosebraugh et al., Ann Neurol. 90: 52-61, 2021), levodopa ethyl ester, levodopa methyl ester, and their salts, which can be rapidly hydrolyzed in the body, typically in an enzyme catalyzed reaction, to form LD, yet can be stored at least for the duration of the intended administration period, for example at least 8 hours, 16 hours, 24 hours, 48 hours, 72 hours, in a reservoir of the drug delivery device. Additional examples of levodopa prodrugs, including LD ester prodrugs, LD amide prodrugs, LD dimeric amide prodrugs, carrier-mediated prodrugs, peptide transport-mediated prodrugs, and cyclic prodrugs, are described in Haddad et al., Molecules 23: 40, 2018, which is incorporated herein by reference.

Preferred modes of administration of the drug-including solid or fluid are via drug delivery devices that are removably secured in the mouth, and which administer the drug into the mouth for a period of at least 4 hours. The drug may be administered at a variable rate, although constant rate administration is preferred. Administration is preferably continuous or semi-continuous.

The administration into the mouth can be for 24 hours daily or it can be limited to the awake period, typically about 16 hours. When limited to the awake period it can be advantageous to administer a morning bolus to more rapidly raise the plasma concentration of the LD than a constant rate administration would. The morning bolus can be delivered, for example, through an orally taken pill or pills of LD and a DDC inhibitor or it can be through administration of a solid or fluid drug into the mouth using the drug devices. Alternatively, the exterior of the drug delivery device may include a drug, such that a bolus of the drug is delivered into the mouth when the device is first inserted into the mouth. The methods of the invention can further include administering into the mouth one or more drugs (e.g., LD and CD) from one or more drug reservoirs residing in the cavity of the mouth including a total volume of 0.1-10 mL of drugs (e.g., 0.1-1.0 mL, 1.0-2.0 mL, 2.0-3.0 mL, 3.0-4.0 mL, 4.0-5.0 mL, mL, 6.0-7.0 mL, 7.0-8.0 mL, 8.0-9.0 mL, or 9.0-10 mL). The methods of the invention can further include administering the one or more drugs (in either solid or fluid form) at a rate in the range of 0.03-1.25 mL/hour (e.g., 0.03-0.10 mL/hour, 0.10-0.20 mL/hour, 0.20-0.30 mL/hour, 0.30-0.40 mL/hour, 0.40-0.50 mL/hour, 0.50-0.60 mL/hour, 0.60-0.70 mL/hour, 0.70-0.80 mL/hour, 0.80-0.90 mL/hour, 0.90-1.0 mL/hour, 1.0-1.1 mL/hour, or 1.1-1.25 mL/hour). The methods of the invention can further include administering the one or more drugs at an average rate of less than 1 mg per hour, 1-10 mg per hour, 10-25 mg per hour, 25-50 mg per hour, 50-75 mg per hour, 75-100 mg per hour, 100-125 mg per hour, or greater than 125 mg per hour. The methods of the invention can further include administering one or more drugs via continuous and/or semi-continuous administration. In a preferred embodiment, the method includes holding the average administration rate constant or near constant for a period of 4, 8, 12, 16, or 24 hours during the day. For example, the volume administered every hour may vary from the average hourly administration rate during the infusion period by less than ±10% or ±20% per hour, or by ±10% or ±20% per 15-minute period. The methods of the invention can further include administering one or more drugs into the mouth using any of the drug delivery devices described herein.

Continuous or semi-continuous administration using the drug delivery devices and formulations of the invention can reduce concentration fluctuations of the therapeutic drug in body fluid, for example in blood, plasma or serum. It can provide, for example, a plasma concentration profile where the difference between peak concentrations and nadir concentrations of the therapeutic drug is less than ±70% of the average concentration through a period in which the drug is administered, for example it can be less than ±50%, less than ±30%, less than ±20%, or less than ±10% of the time averaged concentration over a period of greater than or equal to 4 hours (e.g., 8, 12, 16, or 24 hours).

The methods of the invention can further include treating a disease in a patient, the method including: (a) inserting a drug delivery device into the patient's mouth; (b) starting a drug administration from the device; (c) administering into the patient's mouth one or more drugs, using continuous or semi-continuous administration, for a period of 4 hours to 7 days at an hourly rate in the range of 0.015-1.25 mL/hour or 1-125 mg/hour; and (d) removing the drug delivery device from the mouth; wherein the drug delivery device includes a drug reservoir of 0.1-5 mL volume (e.g., 0.1-1 mL, 0.5-3 mL, or 3-5 mL), and the reservoir includes a solid or fluid including a drug. Optionally, the method may also include the optional step of: (e) stopping the drug delivery from the device. The invention further includes a method wherein steps a, b, c, d and e are performed at least twice over a period of 4 hours to 7 days. The drug may include a total of greater than 1 millimole of LD.

The methods of the invention can further include treating a disease in a patient, the method including: (a) inserting a drug delivery device into the patient's mouth; (b) starting a drug administration from the device; (c) administering into the patient's mouth one or more drugs, using continuous or semi-continuous administration, for a period of 4 hours to 7 days at an hourly rate in the range of 0.015-1.25 mL/hour or 1-125 mg/hour; (d) removing the drug delivery device from the mouth; and (e) stopping the drug delivery from the device, wherein: (1) the drug delivery device includes a drug reservoir of 0.1-5 mL volume (e.g., 0.1-1 mL, 0.5-3 mL, or 3-5 mL), and the reservoir includes a solid or fluid including a drug, and (2) steps a, b, c, d and e are performed at least twice over a period of 4 hours to 7 days. The drug may include a total of greater than 1 millimole of LD.

The methods of the invention can further include treating Parkinson's disease in a patient (including in patients with scores of 4 and 5 on the Hoehn and Yahr scale), the method including: (a) removably inserting a drug delivery device into the patient's mouth, the drug delivery device including a reservoir of 0.1-5 mL volume (e.g., 0.1-1 mL, 0.5-3 mL, or 3-5 mL), and the reservoir including a solid or fluid including a total of greater than 1 millimole of LD; (b) administering into the patient's mouth the solid or fluid for a period of at least 8 hours at an hourly rate in the range of 0.03-1.25 mL/hour or 30-150 mg/hour, such that a circulating plasma LD concentration greater than 400 ng/mL and less than 7,500 ng/mL is continuously maintained for a period of at least 8 hours during the administration; and (c) removing the drug delivery device from the patient's mouth. In certain embodiments, the LD suspension is administered into the mouth at such a rate that a circulating plasma LD concentration greater than 800 ng/mL, 1,200 ng/mL, 1,600 ng/mL, or 2,000 ng/mL (e.g., from 800 to 1,500, from 1,000 to 2,000, from 1,600 to 2,500, or from 1,500 to 3,000 ng/mL, depending upon the condition of the patient) is continuously maintained for a period of at least 2 hours, 3 hours, 4 hours, 8 hours, 16 hours, or 24 hours during the administration. In particular embodiments, the LD suspension is administered into the mouth at such a rate that a circulating plasma LD concentration greater than 400 ng/mL, 800 ng/mL, 1,200 ng/mL, 1,600 ng/mL, or 2,000 is achieved within 60 minutes of the initiation of the infusion. The LD suspension can be administered into the mouth at such a rate that a circulating plasma LD concentration less than 7,500 ng/mL, 5,000 ng/mL, 3,500 ng/mL, 3,000 ng/mL, 2,500 ng/mL, or 2,000 ng/mL is continuously maintained for a period of at least 8 hours during the administration. In particular embodiments, the patient receives an average daily dose of less than 10 mL, 7.5 mL, 5 mL, 3 mL, or 2 mL of the LD suspension. The LD suspension can be administered into the mouth at such a rate that the circulating LD plasma concentration varies by less than ±20%, ±15%, or ±10% from its mean for a period of at least 1 hour, 2 hours, 3 hours, or 4 hours.

The methods of the invention can further include the co-administration of an effective amount of a DDC inhibitor such as benserazide, carbidopa or carbidopa prodrug. Carbidopa can be co-administered as a solid, suspension or emulsion, or as a solution of one of its highly water soluble prodrug salts, exemplified by carbidopa ethyl ester hydrochloride, by carbidopa methyl ester hydrochloride, by carbidopa amide hydrochloride, or by carbidopa phosphate prodrugs (e.g., foscarbidopa, also known as carbidopa 4′-monophosphate, see, e.g., Huters et al., J Org. Chem. 2021 and Rosebraugh et al., Ann Neurol. 90: 52-61, 2021). The molar amount of the co-administered DDC inhibitor can be between one-tenth and one-half of the molar amount of LD, preferably about 1/4^{th}±1/8^{th} of the molar amount of LD. Preparations of the carbidopa prodrugs, recognized to be LD decarboxylase inhibitors, are described, for example, in U.S. Pat. Nos. 3,895,052 and 7,101,912, and Patent Publication Nos. DE2062285A and FR2052983A1. In one particular embodiment, a LD suspension includes a greater than 0.5 M LD (e.g., 0.5±0.1, 0.6±0.1, 0.7±0.1, 0.8±0.2, 1.0±0.3, 1.5±0.5, 2.0±0.5, 0.6±0.3, 0.75±0.25, 1.0±0.5, 1.5±0.5, 2.0±0.5, 2.5±0.5, 3.0±0.5, 3.5±0.5, greater than 1.5, greater than 2, greater than 2.5, or greater than 3.5 moles per liter). In particular embodiments, the LD and the DDC inhibitor are co-administered separately, or are contained in a single solid or fluid and administered into the patient.

The methods of the invention can alleviate a motor or non-motor complication in a patient afflicted with Parkinson's disease, such as tremor, akinesia, bradykinesia, dyskinesia, dystonia, cognitive impairment, and disordered sleep.

### Drug Delivery Devices

The drug delivery devices useful in the methods of the invention are designed to address the requirements for a device that is inserted into the mouth by the patient or caregiver, and that resides in the mouth while it is administering drug, and that can be removed from the mouth by the patient or caregiver. The drug delivery devices typically have a total volume of less than about 10 mL, and preferably less than 7.5, 5.0, or 3.0 mL. The drug delivery devices used in the methods of the invention can be those described in U.S. Publication Nos. US2016-0278899A1 and US2017-0172961A1, each of which is incorporated herein in their entirety.

### Drug Formulations

Formulations of drugs to be delivered via the drug delivery devices using the methods of the invention can be those formulations described in U.S. Publication No. US2017-0172961A1, which is incorporated herein in its entirety. Formulations useful in the methods of the invention are typically suspensions including one or more drugs (which can, e.g., be mostly solid particles) and a liquid (which can, e.g., be an emulsion).

### EXAMPLES

The following examples are meant to illustrate the invention. They are not meant to limit the invention in any way.

### Example 1. Study of Continuous Oral Levodopa

The DopaFuse Delivery System is a novel, intra-oral system that continuously delivers LD/CD at a controlled rate for patients with PD. The DopaFuse System consists of an oral retainer, its case, and a single-use drug container that continuously releases an investigational LD/CD paste in the back of the mouth.

The DopaFuse Delivery System was developed to provide the benefits of continuously delivered LD/CD without the need for a surgical procedure (e.g., with duodenum infusion) and the associated adverse effects. DopaFuse provides continuous oral levodopa via a container, secured by a retainer worn on the teeth, which releases a LD/CD paste at a constant rate. A proof of concept study of continuous oral levodopa therapy (using sips of an oral LD/CD suspension at 5-10 minute intervals) demonstrated that continuous oral levodopa therapy was well tolerated and significantly reduced plasma levodopa variability and Off time in comparison to standard oral LD/CD tablets. As a next step, this study is designed to evaluate the pharmacokinetics, safety, efficacy and tolerability of the system.

The purpose of this study is to evaluate whether the DopaFuse System can reduce the fluctuation of plasma levodopa levels as compared to participants' standard intermittent doses of oral LD/CD tablets (background treatment), and to assess the impact of H. pylori infection on the LD pharmacokinetics (e.g., levodopa variability). An assessment of whether the system is safe, well tolerated, and can relieve motor symptoms will also be made.

### Participants

Approximately 30-35 participants will be screened to achieve an estimated total of 24 participants. Efforts will be made to include 12 participants that are H. pylori negative. H. pylori testing may be performed anytime between Day 1 and Day 15 of the study. The patients have Parkinson's Disease and are on a stable LD/CD treatment who experience at least 2 hours of OFF time per day. Patients will be asked to stop taking COMT inhibitors.

### Dosing

DopaFuse is an oral Paste containing suspended particles of micronized levodopa and carbidopa. Two infusion rates: (i) 50 mg LD/13 mg CD per hour; and (ii) 68 mg LD/17 mg CD per hour will be administered. Treatment regimens will be individualized in order to closely align with the participant's usual dosing quantities and timetable. Over the 15-day active study period, each participant will receive DopaFuse treatment for three days under inpatient observation and 11 days via in-home use. On Day 1, participants will take their usual oral immediate release LD/CD regimen. On Day 2, participants will receive DopaFuse alone. Days 3-15 will involve participants taking their usual initial AM dosing of LD/CD, followed by continuous DopaFuse for the rest of the day. A study summary is shown in Table 2.

### H. pylori Status

The study includes an H. pylori analysis. H. pylori testing will be performed using provided, approved, fecal test kits. Participants will be asked to withhold treatment with H2-receptor antagonists, bismuth or proton pump inhibitors, if such medications can be safely stopped, two weeks prior to testing. Sites will verify participants have not taken antibiotics in the four weeks prior to testing.

There is concern that H. pylori infection may cause alterations in pyloric contraction and duodenitis that could increase levodopa plasma variability. It will be determined whether continuous vs. intermittent oral levodopa delivery is associated with more stable plasma levodopa levels in this population (H. pylori positive) as well, and to assess whether subjects should be tested for H. pylori and treated to further improve the stability of plasma levodopa levels. This study has been designed to have sufficient H. pylori negative participants to ensure the data is not confounded by the presence of H. pylori.

### Pharmacokinetic Analyses

The primary estirnand for the PK study will be the difference in levodopa fluctuation index (FI, Cmax−Cmin)/Caverage) between Day 1 (oral levodopa) and Day 2 (Dopafuse alone). As steady-state levodopa concentrations are not anticipated to be achieved during the first 4 hours of treatment with continuous oral administration, the fluctuation index will be evaluated between hours 4 and 12. Statistical analyses will be performed using the 2-tailed t-test for continuous data and Wilcoxon signed rank test for non-continuous data. Sensitivity analyses will include fluctuation indices in the Per Protocol population. The fluctuation index will also be assessed at hourly intervals for additional sensitivity analyses.

Secondary estimands will be the differences in Coefficient of Variation (CV, standard deviation/mean of levodopa concentrations) and fluctuation index (FI) between Days 1/2 and 1/3, differences at different timepoints in Cmax, Tmax, AUC, and differences in CV and FI by H. pylori status (positive/negative). These will be analyzed in a similar fashion to the primary endpoint.

### Results

This study can demonstrate the importance of H. pylori status on the PK performance of therapies delivered by continuous or semi-continuous intraoral infusion. It is believed that superior results can be achieved if the H. pylori status of subjects is assessed, and H. pylori positive subjects are treated (before or shortly after beginning intraoral infusion), to further reduce drug plasma variability in subjects receiving continuous or semi-continuous intraoral drug infusions.

### Example 2. An 80 mg/mL Baclofen-Comprising Orally Pumpable Suspension for Managing Spastic Conditions in Multiple Sclerosis and Cerebral Palsy

Cocoa butter, an edible oil extracted from cocoa beans, has a typical melting range of about 34° C.-36.5° C., so that it is a solid at room temperature but becomes liquid at body temperature. An 80 mg/mL suspension of Baclofen can be prepared by homogenizing 1.9 g Baclofen with 20 g of cocoa butter. The volume of the suspension at 37±2 ° C. is expected to be about 23.7 mL and the Baclofen concentration is expected to be at 37±2° C. near 80 mg/mL. At 0.04 mL/hour flow rate about 0.64 mL of the solution containing about 0.51 mg of Baclofen could be pumped into the mouth in 16 awake hours.

Subjects are screened for H. pylori infection prior to receiving baclofen treatment. Subjects suffering from an H. pylori infection are treated to eliminate the infection prior to therapy, and eliminate any deleterious impact the infection could have on the pharmacokinetic performance of the therapy. The baclofen formulation is then continuously or semicontinuously administered to the subjects by intraoral infusion. Subjects free of any H. pylori infection can experience a reduced fluctuation index of the circulating baclofen plasma concentrations.

### Example 3. A 50 mg/mL Treprostinil-Comprising Orally Extrudable Paste for Pulmonary Arterial Hypertension Management

Ingredients: Treprostinil (Bio-Techne Minneapolis, MN); L-tyrosine, nominal particle size 20 μm; Poloxamer 188; Miglyol 812 (Peter Cremer, Cincinnati, OH); de-ionized water.

0.8 g Poloxamer 188 can be mixed to homogeneity with 1.5 g water by warming to 60° C. 12 g of L-tyrosine can be added and the mixture can be homogenized, then allowed to age for 10 hours with periodic mixing. 0.8 g of treprostinil can be dissolved in 4.75 g of the medium chain triglyceride Miglyol 812. The treprostinil solution in Miglyol 812 can be mixed with the L-tyrosine -Poloxamer 188-water paste, homogenized and allowed to age for 3 hours with periodic mixing. The resulting paste can be physically stable upon centrifugation at 3000 G and it can be centrifuged to remove trapped air. Its expected density at 23±2° C. is 1.25 g/mL±0.05 g/mL. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.02 mL/hour, 0.32 mL of the paste, containing about 16 mg of treprostinil, would be extruded over 16 awake hours into the mouth.

Subjects are screened for H. pylori infection prior to receiving treprostinil treatment. Subjects suffering from an H. pylori infection are treated to eliminate the infection prior to therapy, and eliminate any deleterious impact the infection could have on the pharmacokinetic performance of the therapy. The treprostinil formulation is then continuously or semicontinuously administered to the subjects by intraoral infusion. Subjects free of any H. pylori infection can experience a reduced fluctuation index of the circulating treprostinil plasma concentrations.

### Example 4. An 80 mg/mL Midoridine-Comprising Orally Extrudable Paste for Managing Spastic Conditions in Multiple Sclerosis and Cerebral Palsy

0.8 g Poloxamer 188 can be mixed to homogeneity with 1.5 g water by warming to 60° C. 1.32 g of Midoridine and 12 g of L-tyrosine can be added and the mixture can be homogenized, then allowed to age for 10 hours with periodic mixing. Next 4.75 g of the medium chain triglyceride Miglyol 812 can be the Baclofen-L-tyrosine-Poloxamer 188-water paste, homogenized and allowed to age for 3 hours with periodic mixing. The resulting paste can be physically stable upon centrifugation at 3000 G and it can be centrifuged to remove trapped air. Its expected density at 23±2° C. is 1.25 g/mL±0.05 g/mL. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.04 mL/hour, 0.64 mL of the paste, containing about 51.2 mg of midoridine, would be extruded over 16 awake hours into the mouth.

Subjects are screened for H. pylori infection prior to receiving midoridine treatment. Subjects suffering from an H. pylori infection are treated to eliminate the infection prior to therapy, and eliminate any deleterious impact the infection could have on the pharmacokinetic performance of the therapy. The midoridine formulation is then continuously or semicontinuously administered to the subjects by intraoral infusion. Subjects free of any H. pylori infection can experience a reduced fluctuation index of the circulating midoridine plasma concentrations.

### Example 5. A 0.5 mg/mL Iloprost-Comprising Orally Extrudable Paste for Management Of Pulmonary Arterial Hypertension

Ingredients: 0.5 g Poloxamer 188 can be mixed to homogeneity with 0.8 g deionized water by warming to about 60° C., and then homogenized with 6.25 g of L-tyrosine (D50 about 20 μm particle size) powder by thorough mixing, aging the mixture at ambient temperature for about 24 hours, and thorough re-mixing. 4 mg of iloprost and 10 mg of BHA (butylated hydroxyanisole) can be dissolved in 2.44 g of Miglyol 812 and the solution can be mixed thoroughly with the L-tyrosine-Poloxamer-water mixture, aging the mixture at ambient temperature for at least 24 hours, then thoroughly re-mixing and again aging for about 24 hours. Next the mixture can be centrifuged for 1 hour at 16,000 G to remove any trapped air. The resulting mixture can be physically stable, i.e. may not phase separate under the centrifugation suggestive of shelf life physical stability for more than 22 months at normal gravity at 23±2° C. Its density can be 1.25±0.05 g/mL at about 25° C. It can be non-pourable at 23±2° C. but can be extruded at 37±2° C. At a continuous extrusion rate of 0.02 mL/hour, 0.36 mL of the paste, containing 0.18 mg of iloprost would be extruded daily into the mouth over 16 awake hours.

Subjects are screened for H. pylori infection prior to receiving iloprost treatment. Subjects suffering from an H. pylori infection are treated to eliminate the infection prior to therapy, and eliminate any deleterious impact the infection could have on the pharmacokinetic performance of the therapy. The iloprost formulation is then continuously or semicontinuously administered to the subjects by intraoral infusion. Subjects free of any H. pylori infection can experience a reduced fluctuation index of the circulating iloprost plasma concentrations.

### Example 6. An 833 mg/mL Carbocisteine Paste for COPD Management

Ingredients: Carbocisteine, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of carbocisteine. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.075 mL/hour, 0.72 mL of the paste containing 1.43 g of carbocisteine would be extruded over 24 hours into the mouth.

Subjects are screened for H. pylori infection prior to receiving carbocisteine treatment. Subjects suffering from an H. pylori infection are treated to eliminate the infection prior to therapy, and eliminate any deleterious impact the infection could have on the pharmacokinetic performance of the therapy. The carbocisteine formulation is then continuously or semicontinuously administered to the subjects by intraoral infusion. Subjects free of any H. pylori infection can experience a reduced fluctuation index of the circulating carbocisteine plasma concentrations.

### Example 7. An 800 mg/mL Erythromycin-Comprising Orally Extrudable Paste for COPD Management

Ingredients: Erythromycin, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of erythromycin. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded.

At a continuous extrusion rate of 0.03 mL/hour, 0.72 mL of the paste containing 576 mg of erythromycin would be extruded over 24 hours into the mouth.

Subjects are screened for H. pylori infection prior to receiving erythromycin treatment. Subjects suffering from an H. pylori infection are treated to eliminate the infection prior to therapy, and eliminate any deleterious impact the infection could have on the pharmacokinetic performance of the therapy. The erythromycin formulation is then continuously or semicontinuously administered to the subjects by intraoral infusion. Subjects free of any H. pylori infection can experience a reduced fluctuation index of the circulating erythromycin plasma concentrations.

### Example 8. An 30 mg/mL Tizanidine-Comprising Orally Extrudable Paste for Managing Spastic Conditions in Multiple Sclerosis and Cerebral Palsy

0.8 g Poloxamer 188 can be mixed to homogeneity with 1.5 g water by warming to 60° C. 0.5 g of Tizanidine and 12 g of L-tyrosine can be added and the mixture can be homogenized, then allowed to age for 10 hours with periodic mixing. Next 4.75 g of the medium chain triglyceride Miglyol 812 can be the Baclofen-L-tyrosine-Poloxamer 188-water paste, homogenized and allowed to age for 3 hours with periodic mixing. The resulting paste can be physically stable upon centrifugation at 3000 G and it can be centrifuged to remove trapped air. Its expected density at 23±2° C. is 1.25 g/mL±0.05 g/mL. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.04 mL/hour, 0.64 mL of the paste, containing about 20 mg of tizadinide, would be extruded over 16 awake hours into the mouth.

Subjects are screened for H. pylori infection prior to receiving tizadinide treatment. Subjects suffering from an H. pylori infection are treated to eliminate the infection prior to therapy, and eliminate any deleterious impact the infection could have on the pharmacokinetic performance of the therapy. The tizadinide formulation is then continuously or semicontinuously administered to the subjects by intraoral infusion. Subjects free of any H. pylori infection can experience a reduced fluctuation index of the circulating tizadinide plasma concentrations.

### Example 9. An 800 mg/mL Flavoxate-Comprising Orally Extrudable Paste for Urinary Urge And Incontinence (“Overactive Bladder”) Management

Ingredients: Flavoxate, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of flavoxate. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.04 mL/hour, 0.96 mL of the paste containing 768 mg of flavoxate would be extruded over 24 hours into the mouth.

Subjects are screened for H. pylori infection prior to receiving flavoxate treatment. Subjects suffering from an H. pylori infection are treated to eliminate the infection prior to therapy, and eliminate any deleterious impact the infection could have on the pharmacokinetic performance of the therapy. The flavoxate formulation is then continuously or semicontinuously administered to the subjects by intraoral infusion. Subjects free of any H. pylori infection can experience a reduced fluctuation index of the circulating flavoxate plasma concentrations.

### Example 10. A 1.14 g/mL Magnesium Carbonate Comprising Orally Extrudable Paste E.g. For Managing a Neurological Disorder Like Alzheimer'S Disease or Parkinson'S Disease

Ingredients: Magnesium carbonate, about 20 μm nominal particle size; Poloxamer 188; Miglyol 812 (Peter Cremer, Cincinnati, OH); de-ionized water.

0.8 g Poloxamer 188 can be dissolved in 5 g water then homogenized with 20 g of magnesium carbonate. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.2 mL/hour, optionally from 1.2 mL paste containing reservoirs replaced every 6 hours, 4.8 mL of the paste containing 5.6 g or about 0.067 moles of magnesium carbonate would be extruded over 24 hours into the mouth. With a pair of bilateral devices 11.2 g or about 0.132 moles of magnesium carbonate would be extruded daily into the mouth.

Subjects are screened for H. pylori infection prior to receiving magnesium carbonate treatment. Subjects suffering from an H. pylori infection are treated to eliminate the infection prior to therapy, and eliminate any deleterious impact the infection could have on the pharmacokinetic performance of the therapy. The magnesium carbonate formulation is then continuously or semicontinuously administered to the subjects by intraoral infusion. Subjects free of any H. pylori infection can experience a reduced fluctuation index of the circulating magnesium plasma concentrations.

### Example 11. An 800 mg/mL Trimebutine-Comprising Orally Extrudable Paste for Irritable Bowel Syndrome Management

Ingredients: Trimebutine, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of trimebutine. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.03 mL/hour, 0.72 mL of the paste containing 576 mg of trimebutine would be extruded over 24 hours into the mouth.

Subjects are screened for H. pylori infection prior to receiving trimebutine treatment. Subjects suffering from an H. pylori infection are treated to eliminate the infection prior to therapy, and eliminate any deleterious impact the infection could have on the pharmacokinetic performance of the therapy. The trimebutine formulation is then continuously or semicontinuously administered to the subjects by intraoral infusion. Subjects free of any H. pylori infection can experience a reduced fluctuation index of the circulating trimebutine plasma concentrations.

### Example 12. An 800 mg/mL Curcumin-Comprising Orally Extrudable Paste for Cancer Therapy

Ingredients: Curcumin, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of curcumin. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.1 mL/hour, 2.4 mL of the paste containing 1.92 g of curcumin would be extruded over 24 hours into the mouth.

Subjects are screened for H. pylori infection prior to receiving curcumin treatment. Subjects suffering from an H. pylori infection are treated to eliminate the infection prior to therapy, and eliminate any deleterious impact the infection could have on the pharmacokinetic performance of the therapy. The curcumin formulation is then continuously or semicontinuously administered to the subjects by intraoral infusion. Subjects free of any H. pylori infection can experience a reduced fluctuation index of the circulating curcumin plasma concentrations.

### Example 13. An 800 mg/mL Curcumin-Analog EF31-Comprising Orally Extrudable Paste for Cancer Therapy

Ingredients: Curcumin-analog EF31, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of Curcumin-analog EF31. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.1 mL/hour, 2.4 mL of the paste containing 1.92 g of curcumin-analog EF31 would be extruded over 24 hours into the mouth.

Subjects are screened for H. pylori infection prior to receiving EF31 treatment. Subjects suffering from an H. pylori infection are treated to eliminate the infection prior to therapy, and eliminate any deleterious impact the infection could have on the pharmacokinetic performance of the therapy. The EF31 formulation is then continuously or semicontinuously administered to the subjects by intraoral infusion. Subjects free of any H. pylori infection can experience a reduced fluctuation index of the circulating EF31 plasma concentrations.

### Example 14. An 800 mg/mL Curcumin-Analog UBS109-Comprising Orally Extrudable Paste for Cancer Therapy.

Ingredients: Curcumin-analog UBS109, about 20 μm nominal particle size; Poloxamer 188; Miglyol 812 (Peter Cremer, Cincinnati, OH); de-ionized water.

0.8 g Poloxamer 188 can be dissolved in 1.5 g water then homogenized with 10 g of curcumin-analog UBS109. The mixture can be allowed to age for 10 hours at 23±2° C. with periodic mixing, and then homogenized by thorough mixing with 4.75 g of the medium chain triglyceride Miglyol 812. The mixture can be allowed to age for at least 12 hours with periodic mixing. The resulting paste can be physically stable upon centrifugation at 3000 G and it can be centrifuged to remove trapped air. The paste is expected to be soft, compliant, and easy to mechanically deform and to retain at 23±2° C. its shape after deformation. The paste is not expected to be pourable at 23±2° C., but at 37±2° C. it could be extruded. At a continuous extrusion rate of 0.1 mL/hour, 2.4 mL of the paste containing 1.92 g of curcumin-analog UBS109 would be extruded over 24 hours into the mouth.

Subjects are screened for H. pylori infection prior to receiving UBS109 treatment. Subjects suffering from an H. pylori infection are treated to eliminate the infection prior to therapy, and eliminate any deleterious impact the infection could have on the pharmacokinetic performance of the therapy. The UBS109 formulation is then continuously or semicontinuously administered to the subjects by intraoral infusion. Subjects free of any H. pylori infection can experience a reduced fluctuation index of the circulating UBS109 plasma concentrations.

### Other Embodiments

Various modifications and variations of the described invention will be apparent to those skilled in the art without departing from the scope and spirit of the invention. Although the invention has been described in connection with specific embodiments, it should be understood that the invention as claimed 5 should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of administering a pharmaceutical composition comprising at least one drug to a subject, the method comprising:
(i) providing a subject identified as being free of infection by H. pylori;
(ii) inserting a drug delivery device into a mouth of the subject, the drug delivery device comprising a drug reservoir comprising the pharmaceutical composition comprising at least one drug; and
(iii) continuously or semicontinuously administering the pharmaceutical composition into the mouth of the subject at a rate between 0.001 mL/hour and 1.25 mL/hour.

2. A method of administering a pharmaceutical composition comprising at least one drug to a subject, the method comprising:
(i) providing a subject, wherein the subject has been tested for an infection by H. pylori, and, if infected, treated for the H. pylori infection;
(ii) inserting a drug delivery device into a mouth of the subject, the drug delivery device comprising a drug reservoir comprising the pharmaceutical composition comprising at least one drug; and
(iii) continuously or semicontinuously administering the pharmaceutical composition into the mouth of the subject at a rate between 0.001 mL/hour and 1.25 mL/hour.

3. The method of claim 1 or 2, wherein the drug delivery device is removably attached to an intraoral surface of the subject.

4. The method of any one of claims 1-3, wherein the pharmaceutical composition is administered to the subject over a delivery period of 4, 8, 16, 24, or more hours.

5. The method of claim 4, wherein the rate of delivery of said pharmaceutical composition during each hour of the delivery period is within +/−25% or +/−10% of the average rate of delivery during the delivery period.

6. The method of any one of claims 1-5, wherein the half-life of said drug is less than 8 hours, 6 hours, 4 hours, 2 hours, 1 hour, 30 min, 20 min or 10 min.

7. The method of any one of claims 4-6, wherein the fluctuation index of the drug is less than or equal to 2.0, 1.5, 1.0, 0.75, 0.50, 0.25, or 0.15 during the delivery period.

8. The method of claim 7, wherein the fluctuation index of the drug is less than the average fluctuation index observed for subjects of the same condition and receiving the same therapy but having an H. pylori infection.

9. The method of any one of claims 1-8, wherein the method comprises administering the drug to the subject from a drug delivery device for a delivery period of not less than about 4 hours and not more than about 7 days.

10. The method of any one of claims 1-9, wherein the drug reservoir comprises a volume of the pharmaceutical composition, and the method further comprises oral administration at a rate in the range of from about 15 μL per hour to about 1.25 mL per hour during the delivery period.

11. The method of any one of claims 1-10, wherein the method comprises oral administration at a rate in the range of from about 0.015 mL/hour to about 0.25 mL/hour.

12. The method of any one of claims 1-11, wherein the method comprises oral administration at a rate in the range of from about 0.25 mL/hour to about 0.5 mL/hour; from about 0.5 mL/hour to about mL/hour; or from about 0.75 mL/hour to about 1.0 mL/hour.

13. The method of any one of claims 1-12, wherein the drug is administered to the subject at an average rate of not less than 0.01 mg per hour and not more than 250 mg per hour.

14. The method of any one of claims 1-13, wherein the drug is administered to the subject at an hourly rate in the range of 0.01 mg per hour to 1 mg per hour, 1 mg per hour to 10 mg per hour, 10 mg per hour to 100 mg per hour, or 100 mg per hour to 250 mg per hour.

15. The method of any one of claims 1-14, wherein the pharmaceutical composition is administered to the subject at least once every 60 minutes.

16. The method of any one of claims 1-14, wherein the pharmaceutical composition is administered to the subject at least once every 30 minutes.

17. The method of any one of claims 1-14, wherein the pharmaceutical composition is administered to the subject at least once every 15 minutes.

18. The method of any one of claims 1-14, wherein the pharmaceutical composition is administered to the subject continuously.

19. The method of any one of claims 1-18, further comprising treating a disease in the subject, wherein the disease is mucositis, an allergy, an immune disease, anesthesia, a bacterial infection, cancer, pain, organ transplantation, disordered sleep, epilepsy or a seizure, anxiety, a mood disorder, post-traumatic stress disorder, arrhythmia, hypertension, heart failure, spasticity, or diabetic nephropathy.

20. The method of any one of claims 1-18, further comprising treating a disease in the subject, wherein the disease is multiple sclerosis, cerebral palsy, spasticity, neurogenic orthostatic hypotension, Wilson's disease, cystinuria, rheumatoid arthritis, Alzheimer's disease, Type-1 Gaucher disease, Type C Niemann-Pick disease, eosinophilic gastroenteritis, chronic mastocytosis, ulcerative colitis, gastro-oesophageal reflux, gastroenteritis, hyperemesis gravidarum, glioblastoma multiformae, anaplastic astrocytoma, pulmonary hypertension, coronary heart disease, congestive heart failure, angina, Type 2 diabetes, COPD, asthma, irritable bowel syndrome, overactive bladder, or urinary urge incontinence.

21. The method of any one of claims 1-18, wherein the pharmaceutical composition comprises one or more drugs selected from levodopa or a levodopa prodrug, baclofen or a baclofen prodrug, pyridostigmine or a pyridostigmine prodrug, pilocarpine or a pilocarpine prodrug, furosemide or a furosemide prodrug, methylphenidate, a prostaglandin, prostacyclin, treprostinil, beraprost, nimodipine, and testosterone.

22. The method of any one of claims 1-18, wherein (a) the subject has Parkinson's disease, (b) the drug reservoir comprises levodopa or a levodopa prodrug, and (c) the method comprises administering into the subject's mouth the levodopa or a levodopa prodrug for a period of at least 4 hours at an hourly rate in the range of 30 mg/hour to 150 mg/hour.

23. The method of claim 22, wherein a circulating plasma levodopa concentration greater than 1,200 ng/mL and less than 2,500 ng/mL is continuously maintained for a period of at least 4 hours during the administration.

24. The method of claim 23, wherein the subject has a score of 4 and 5 on the Hoehn and Yahr scale.

25. The method of any one of claims 1-18, wherein the method comprises treating spasticity in a subject in need thereof, wherein the drug is baclofen or a baclofen prodrug.

26. The method of any one of claims 1-18, wherein the method comprises treating myasthenia gravis in a subject in need thereof, wherein the drug is pyridostigmine or a pyridostigmine prodrug.

27. The method of any one of claims 1-18, wherein the method comprises treating dry mouth in a subject in need thereof, wherein the drug is pilocarpine or a pilocarpine prodrug.

28. The method of any one of claims 1-18, wherein the method comprises treating edema or high blood pressure in a subject in need thereof, wherein the drug is furosemide or a furosemide prodrug.

29. The method of any one of claims 1-28, wherein during the administration the circulating drug plasma concentration varies by less than +/−20% or +/−10% from its mean for a period of at least 1, 2, or 4 hours.

30. The method of any one of claims 1-29, wherein an efficacious circulating plasma concentration of the drug is continuously maintained for a period of at least 8 hours during the administration.

31. The method of any one of claims 1-30, wherein the drug delivery device is configured to be removably inserted in a subject's mouth and for continuous or semi-continuous intraoral administration of the drug, the device comprising:
(i) a fastener to removably secure the drug delivery device to a surface of the subject's mouth;
(ii) an electrical or mechanical pump; and
(iii) a drug reservoir containing the pharmaceutical composition, the volume of the drug reservoir being from 0.1 mL to 5 mL.

32. Use of levodopa or a levodopa prodrug in the treatment of Parkinson's disease, wherein the levodopa or levodopa prodrug is administered via a drug delivery device, the use comprising:
(i) providing a subject identified as being free of infection by H. pylori;
(ii) inserting the drug delivery device into a mouth of the subject, the drug delivery device comprising a drug reservoir comprising a pharmaceutical composition comprising the levodopa or levodopa prodrug; and
(iii) continuously or semicontinuously administering the levodopa or levodopa prodrug into the mouth of the subject at a rate between 0.001 mL/hour and 1.25 mL/hour.

33. Use of levodopa or a levodopa prodrug in the treatment of Parkinson's disease, wherein the levodopa or levodopa prodrug is administered via a drug delivery device, the use comprising:
(i) providing a subject, wherein the subject has been tested for an infection by H. pylori, and, if infected, treated for the H. pylori infection;
(ii) inserting the drug delivery device into a mouth of the subject, the drug delivery device comprising a drug reservoir comprising a pharmaceutical composition comprising the levodopa or levodopa prodrug; and
(iii) continuously or semicontinuously administering the levodopa or levodopa prodrug into the mouth of the subject at a rate between 0.001 mL/hour and 1.25 mL/hour.

34. The use according to claim 32 or 33, wherein the use comprises administering into the subject's mouth the levodopa or levodopa prodrug for a period of at least 4 hours at an hourly rate in the range of 30 mg/hour to 150 mg/hour.

35. The use according to claim 34, wherein a circulating plasma levodopa concentration greater than 1,200 ng/mL and less than 2,500 ng/mL is continuously maintained for a period of at least 4 hours during the administration.

36. The use according to claim 35, wherein the subject has a score of 4 and 5 on the Hoehn and Yahr scale.

37. The use according to any one of claims 32-36, wherein the pharmaceutical composition further comprises carbidopa or a carbidopa prodrug.

38. The use according to any one of claims 32-37, wherein the pharmaceutical composition further comprises benserazide.

39. Use of baclofen or a baclofen prodrug in the treatment of spasticity, wherein the baclofen or baclofen prodrug is administered via a drug delivery device, the use comprising:
(i) providing a subject identified as being free of infection by H. pylori;
(ii) inserting the drug delivery device into a mouth of the subject, the drug delivery device comprising a drug reservoir comprising a pharmaceutical composition comprising the baclofen or baclofen prodrug; and
(iii) continuously or semicontinuously administering the baclofen or baclofen prodrug into the mouth of the subject at a rate between 0.001 mL/hour and 1.25 mL/hour.

40. Use of baclofen or a baclofen prodrug in the treatment of spasticity, wherein the baclofen or baclofen prodrug is administered via a drug delivery device, the use comprising:
(i) providing a subject, wherein the subject has been tested for an infection by H. pylori, and, if infected, treated for the H. pylori infection;
(ii) inserting the drug delivery device into a mouth of the subject, the drug delivery device comprising a drug reservoir comprising a pharmaceutical composition comprising the baclofen or baclofen prodrug; and
(iii) continuously or semicontinuously administering the baclofen or baclofen prodrug into the mouth of the subject at a rate between 0.001 mL/hour and 1.25 mL/hour.

41. The use according to claim 39 or 40, wherein the subject has multiple sclerosis or a spinal cord injury.

42. Use of pyridostigmine or a pyridostigmine prodrug in the treatment of myasthenia gravis, wherein the pyridostigmine or pyridostigmine prodrug is administered via a drug delivery device, the use comprising:
(i) providing a subject identified as being free of infection by H. pylori;
(ii) inserting the drug delivery device into a mouth of the subject, the drug delivery device comprising a drug reservoir comprising a pharmaceutical composition comprising the pyridostigmine or pyridostigmine prodrug; and
(iii) continuously or semicontinuously administering the pyridostigmine or pyridostigmine prodrug into the mouth of the subject at a rate between 0.001 mL/hour and 1.25 mL/hour.

43. Use of pyridostigmine or a pyridostigmine prodrug in the treatment of myasthenia gravis, wherein the pyridostigmine or pyridostigmine prodrug is administered via a drug delivery device, the use comprising:
(i) providing a subject, wherein the subject has been tested for an infection by H. pylori, and, if infected, treated for the H. pylori infection;
(ii) inserting the drug delivery device into a mouth of the subject, the drug delivery device comprising a drug reservoir comprising a pharmaceutical composition comprising the pyridostigmine or pyridostigmine prodrug; and
(iii) continuously or semicontinuously administering the pyridostigmine or pyridostigmine prodrug into the mouth of the subject at a rate between 0.001 mL/hour and 1.25 mL/hour.

44. Use of pilocarpine or a pilocarpine prodrug in the treatment of dry mouth, wherein the pilocarpine or pilocarpine prodrug is administered via a drug delivery device, the use comprising:
(i) providing a subject identified as being free of infection by H. pylori;
(ii) inserting the drug delivery device into a mouth of the subject, the drug delivery device comprising a drug reservoir comprising a pharmaceutical composition comprising the pilocarpine or pilocarpine prodrug; and
(iii) continuously or semicontinuously administering the pilocarpine or pilocarpine prodrug into the mouth of the subject at a rate between 0.001 mL/hour and 1.25 mL/hour.

45. Use of pilocarpine a pilocarpine prodrug in the treatment of dry mouth, wherein the pilocarpine or pilocarpine prodrug is administered via a drug delivery device, the use comprising:
(i) providing a subject, wherein the subject has been tested for an infection by H. pylori, and, if infected, treated for the H. pylori infection;
(ii) inserting the drug delivery device into a mouth of the subject, the drug delivery device comprising a drug reservoir comprising a pharmaceutical composition comprising the pilocarpine or pilocarpine prodrug; and
(iii) continuously or semicontinuously administering the pilocarpine or pilocarpine prodrug into the mouth of the subject at a rate between 0.001 mL/hour and 1.25 mL/hour.

46. The use according to claim 44 or 45, wherein the dry mouth is associated with Sjogren's syndrome or radiation therapy.

47. Use of furosemide or a furosemide prodrug in the treatment of high blood pressure or edema, wherein the furosemide or furosemide prodrug is administered via a drug delivery device, the use comprising:
(i) providing a subject identified as being free of infection by H. pylori;
(ii) inserting the drug delivery device into a mouth of the subject, the drug delivery device comprising a drug reservoir comprising a pharmaceutical composition comprising the furosemide or furosemide prodrug; and
(iii) continuously or semicontinuously administering the furosemide or furosemide prodrug into the mouth of the subject at a rate between 0.001 mL/hour and 1.25 mL/hour.

48. Use of furosemide or a furosemide prodrug in the treatment of high blood pressure or edema, wherein the furosemide or furosemide prodrug is administered via a drug delivery device, the use comprising:
(i) providing a subject, wherein the subject has been tested for an infection by H. pylori, and, if infected, treated for the H. pylori infection;
(ii) inserting the drug delivery device into a mouth of the subject, the drug delivery device comprising a drug reservoir comprising a pharmaceutical composition comprising the furosemide or furosemide prodrug; and
(iii) continuously or semicontinuously administering the furosemide or furosemide prodrug into the mouth of the subject at a rate between 0.001 mL/hour and 1.25 mL/hour.

49. The use according to claim 47 or 48, wherein the edema is associated with heart failure, kidney disease, or liver disease.

50. The use according to any one of claims 32-49, wherein the drug delivery device is removably attached to an intraoral surface of the subject.

51. The use according to any one of claims 32-50, wherein the pharmaceutical composition is administered to the subject over a delivery period of 4, 8, 16, 24, or more hours.

52. The use according to claim 51, wherein the rate of delivery of said pharmaceutical composition during each hour of the delivery period is within +/−25% or +/−10% of the average rate of delivery during the delivery period.

53. The use according to any one of claims 32-52, wherein the half-life of the levodopa, baclofen, pyridostigmine, pilocarpine, furosemide, or a prodrug thereof is less than 8 hours, 6 hours, 4 hours, 2 hours, 1 hour, 30 minutes, 20 minutes or 10 minutes.

54. The use according to any one of claims 51-53, wherein the fluctuation index of the levodopa, baclofen, pyridostigmine, pilocarpine, furosemide, or a prodrug thereof is less than or equal to 2.0, 1.5, 1.0, 0.75, 0.50, 0.25, or 0.15 during the delivery period.

55. The use according to claim 54, wherein the fluctuation index of the levodopa, baclofen, pyridostigmine, pilocarpine, furosemide, or a prodrug thereof is less than the average fluctuation index observed for subjects with the same condition and receiving the same therapy but having an H. pylori infection.

56. The use according to any one of claims 32-55, wherein the use comprises administering the levodopa, baclofen, pyridostigmine, pilocarpine, furosemide, or a prodrug thereof to the subject from a drug delivery device for a delivery period of not less than about 4 hours and not more than about 7 days.

57. The use according to any one of claims 32-56, wherein the drug reservoir comprises a volume of the pharmaceutical composition, and the method further comprises oral administration at a rate in the range of from 15 μL per hour to about 1.25 mL per hour during the delivery period.

58. The use according to any one of claims 32-57, wherein the use comprises oral administration at a rate in the range of from about 0.015 mL/hour to about 0.25 mL/hour.

59. The use according to any one of claims 32-58, wherein the use comprises oral administration at a rate in the range of from about 0.25 mL/hour to about 0.5 mL/hour; from about 0.5 mL/hour to about mL/hour; or from about 0.75 mL/hour to about 1.0 mL/hour.

60. The use according to any one of claims 32-59, wherein the levodopa, baclofen, pyridostigmine, pilocarpine, furosemide, or a prodrug thereof is administered to the subject at an average rate of not less than 0.01 mg per hour and not more than 250 mg per hour.

61. The use according to any one of claims 32-60, wherein the levodopa, baclofen, pyridostigmine, pilocarpine, furosemide, or a prodrug thereof is administered to the subject at an hourly rate in the range of 0.01 mg per hour to 1 mg per hour, 1 mg per hour to 10 mg per hour, 10 mg per hour to 100 mg per hour, or 100 mg per hour to 250 mg per hour.

62. The use according to any one of claims 32-61, wherein the pharmaceutical composition is administered to the subject at least once every 60 minutes.

63. The use according to any one of claims 32-61, wherein the pharmaceutical composition is administered to the subject at least once every 30 minutes.

64. The use according to any one of claims 32-61, wherein the pharmaceutical composition is administered to the subject at least once every 15 minutes.

65. The use according to any one of claims 32-61, wherein the pharmaceutical composition is administered to the subject continuously.

66. The use according to any one of claims 32-65, wherein during the administration the circulating levodopa, baclofen, pyridostigmine pilocarpine, furosemide, or a prodrug thereof, plasma concentration varies by less than +/−20% or +/−10% from its mean for a period of at least 1, 2, or 4 hours.

67. The use according to any one of claims 32-66, wherein an efficacious circulating plasma concentration of the levodopa, baclofen, pyridostigmine, pilocarpine, furosemide, or a prodrug thereof is continuously maintained for a period of at least 8 hours during the administration.

68. The use according to any one of claims 32-67, wherein the drug delivery device is configured to be removably inserted in a subject's mouth and for continuous or semi-continuous intraoral administration of the levodopa, baclofen, pyridostigmine, pilocarpine, furosemide, or a prodrug thereof, the device comprising:
(i) a fastener to removably secure the drug delivery device to a surface of the subject's mouth;
(ii) an electrical or mechanical pump; and
(iii) a drug reservoir containing the pharmaceutical composition, the volume of the drug reservoir being from 0.1 mL to 5 mL.
